(19) United States
(12) Patent Application Publication
(10) Pub. No.: US 2018/0104167 A1
(43) Pub. Date: Apr. 19, 2018

(54) Hair Dye Removal Composition and Methods of Preparing

(71) Applicant: Jamilah, LLC, Phoenix, AZ (US)

(72) Inventor: Mazen Alsharbaji, Gilbert, AZ (US)

(73) Assignee: Jamilah, LLC, Phoenix, AZ (US)

(21) Appl. No.: 15/846,032

(22) Filed: Dec. 18, 2017

Related U.S. Application Data
(63) Continuation-in-part of application No. 15/055,780, filed on Feb. 29, 2016, now abandoned.

(51) Int. Cl. A61K 8/44; A61K 8/92; A61K 8/34; A61K 8/81; A61K 8/368; A61K 8/41; A61Q 5/08; A61Q 5/02; A61Q 5/12

(52) U.S. Cl. CPC A61K 8/44; A61K 8/447; A61K 8/922; A61K 8/345; A61Q 5/12; A61K 8/368; A61K 8/41; A61Q 5/08; A61Q 5/02; A61K 8/8152

(57) ABSTRACT

A hair dye removal composition includes a cleansing agent, a humectant, a rheology modifier, arginine, cysteine, and aminomethyl propanol. The cleansing agent includes between approximately 60 wt % to approximately 80 wt % of the hair conditioning composition, the humectant includes between approximately 10 wt % to approximately 20 wt % of the hair conditioning composition, the rheology modifier includes between approximately 2 wt % and approximately 10 wt % of hair conditioning composition, the arginine includes between approximately 0.05 wt % and approximately 0.8 wt %, the cysteine includes between approximately 0.02 wt % and approximately 0.08 weight %, and the aminomethyl propanol includes between approximately 1 wt % and approximately 6 wt % of the hair conditioning composition. The cleansing agent may include sulfated castor oil, the humectant may include propylene glycol, and the rheology modifier may include acrylates/steareth-20 methacrylate copolymer.

### CROSS REFERENCE TO RELATED APPLICATIONS

This application is a continuation in part of U.S. application Ser. No. 15/055,780 entitled “Hair Cleansing, Conditioning, or Treatment Composition and Methods of Preparing” to Mazen Alsharbaji, filed on Feb. 29, 2016, the content of which is hereby incorporated by reference in its entirety.

### BACKGROUND

### 1. Technical Field

Aspects of this document relate generally to hair cleansing, conditioning, hair damage repair, or treatment compositions.

### 2. Background Art

Various compositions are used in the treatment of hair for the purposes of cleansing and/or conditioning the hair.

### SUMMARY

According to one aspect, a hair dye removal composition may comprise a cleansing agent, a humectant, a rheology modifier, arginine, cysteine, and aminomethyl propanol.

Various implementations and embodiments may comprise one or more of the following. The cleansing agent may comprise between approximately 60 wt % to approximately 80 wt % of the hair dye removal composition, the humectant may comprise between approximately 10 wt % to approximately 20 wt % of the hair dye removal composition, the rheology modifier may comprise between approximately 2 wt % and approximately 10 wt % of hair dye removal composition, the arginine may comprise between approximately 0.05 wt % and approximately 0.8 wt % of the hair dye removal composition, the cysteine may comprise between approximately 0.02 wt % and approximately 0.08 wt % of the hair dye removal composition, and the aminomethyl propanol may comprise between approximately 1 wt % and approximately 6 wt % of the hair dye removal composition. The hair dye removal composition may further comprise salicylic acid, the salicylic acid comprising between approximately 1 wt % and approximately 6 wt % of the hair dye removal composition.

The cleansing agent may comprise between approximately 68 wt % and approximately 74 wt % of the hair dye removal composition, the humectant may comprise between approximately 14 wt % to approximately 17 wt % of the hair dye removal composition, the rheology modifier may comprise between approximately 4 wt % and approximately 6 wt % of hair dye removal composition, the aminomethyl propanol may comprise between approximately 2 wt % and approximately 4 wt % of the hair dye removal composition, the arginine may comprise between approximately 0.03 wt % and approximately 0.1 wt % of the hair dye removal composition, the cysteine may comprise between approximately 0.01 wt % and approximately 0.1 wt % of the hair dye removal composition, and the salicylic acid may comprise between approximately 2 wt % and approximately 4 wt % of the hair dye removal composition. The cleansing agent maycomprise sulfated castor oil, the humectant may comprise propylene glycol, and the rheology modifier may comprise acrylates/steareth-20 methacrylatecopolymer.

The hair dye removal composition may further comprise water and a preservative, the water comprising between approximately 1 wt % and approximately 3 wt % of the hair dye removal composition and the preservative comprising between approximately 0.01 wt % and approximately 0.1 wt % of the hair dye removal composition. The hair dye removal composition may further comprise a fragrance, the fragrance comprising between approximately 0.05 wt % and approximately 0.25 wt % of the hair conditioning composition.

According to another aspect, a hair dye removal composition may comprise oil, humectant, arginine, cysteine, and a pH buffer selected from the group consisting of 2-amino-2-methyl-1-propanol and aminomethyl propanol, the oil comprising greater than 50 wt % of the hair dye removal composition, the humectant comprising less than 20 wt % of the hair dye removal composition, the arginine comprising less than 5 wt % of the hair dye removal composition, the cysteine comprising less than 1 wt % and the pH buffer comprising less than 5 wt % of the hair dye removal composition.

Various implementations and embodiments may comprise one or more of the following. The hair dye removal composition may further comprise a rheology modifier and an acid, the rheology modifier comprising less than 10 wt % of the hair dye removal composition and the acid comprising less than 5 wt % of the hair dye removal composition. The rheology modifier may comprise acrylates/steareth-20 methacrylate copolymer and the acid may comprise salicylic acid. The oil may comprise between approximately 60 wt % to approximately 80 wt % of the hair dye removal composition, the humectant may comprise between approximately 10 wt % to approximately 20 wt % of the hair dye removal composition, and the aminomethyl propanol may comprise between approximately 2 wt% and approximately 4 wt % of the hair dye removal composition.

The oil may comprise between approximately 68 wt % and approximately 74 wt % of the hair dye removal composition, the humectant may comprise between approximately 14 wt % to approximately 17 wt % of the hair dye removal composition, and the aminomethyl propanol may comprise between approximately 2.5 wt % and approximately 3.5 wt % of the hair dye removal composition. The oil may comprise sulfated castor oil, the humectant may comprise propylene glycol, and the pH buffer may comprise amp-95 2-amino-2-methyl-1-propanol. The hair dye removal composition may further comprising water, a fragrance, and ethylenediaminetetraacetic acid (EDTA), the water comprising between approximately 1 wt % and approximately 3 wt % of the hair dye removal composition, the fragrance comprising less than 1 wt % of the hair dye removal composition, and the EDTA comprising less than 1 wt % of the hair dye removal composition.

According to one aspect, a method of preparing a hair dye removal composition, may comprise premixing arginine and cysteine in water, heating a cleansing agent to between approximately 45 degrees Celsius and approximately 60 degrees Celsius, mixing a humectant and a rheology modifier with the cleansing agent; mixing aminomethyl propanol with the cleaning agent, the humectant, and the rheology modifier and mixing the premixed arginine and cysteine with the cleansing agent, the humectant, the rheology modifier, and the aminomethyl propanol at a temperature that is below 60 degrees Celsius.

Various implementations and embodiments may comprise one or more of the following. Mixing the humectant and the rheology modifier with the cleansing agent may comprise premixing a first portion of salicylic acid and a first portion of the humectant before mixing the first portion of the salicylic acid and the first portion of the humectant with the cleansing agent, premixing a second portion of the salicylic acid and a second portion of the humectant before mixing the second portion of the salicylic acid and the second portion of the humectant with the cleansing agent, the first portion of the salicylic acid, and the first portion of the humectant, and adding the rheology modifier to the first and second portions of the salicylic acid, the first and second portions of the humectant, and the cleansing agent at a maximum rate of 30 kg/minute.

The method may further comprise mixing ethylenediaminetetraacetic acid (EDTA) with the cleansing agent for at least 15 minutes before mixing the first portion of the humectant and the first portion of the salicylic acid to the cleansing agent and adding water after adding the rheology modifier and before mixing the aminomethyl propanol with the cleansing agent. The method may further comprise mixing the cleansing agent, the EDTA, the first and second portions of the humectant, the first and second portions of the salicylic acid, the rheology modifier, and the water for at least 75 minutes before mixing the aminomethyl propanol with the cleansing agent.

The arginine may comprise between approximately 0.1 wt % and approximately 0.5 wt % of the hair dye removal composition, the cysteine may comprise between approximately 0.01 wt % and approximately 0.1 wt % of the hair dye removal composition, the cleansing agent may comprise between approximately 65 wt % and approximately 75 wt % of the hair dye removal composition, the EDTA may comprise less than 1 wt % of the hair dye removal composition, the first portion of the humectant may comprise between approximately 5 wt % and approximately 9 wt % of the hair dye removal composition, the first portion of the salicylic acid may comprise between approximately 1.75 wt % and approximately 2.75 wt % of the hair dye removal composition, the second portion of the humectant may comprise between approximately 5 wt % and approximately 9 wt % of the hair dye removal composition, the second portion of the salicylic acid may comprise between approximately 1.75 wt % and approximately 2.75 wt % of the hair dye removal composition, the rheology modifier may comprise between approximately 3 wt % and approximately 7 wt % of the hair dye removal composition, and the aminomethyl propanol may comprise between approximately 2 wt % and approximately 4 wt % of the hair dye removal composition. The cleansing agent may comprise sulfated castor oil, the humectant may comprise propylene glycol, and the rheology modifier may comprise acrylates/steareth-20 methacrylate copolymer.

The foregoing and other aspects, features, and advantages will be apparent to those artisans of ordinary skill in the art from the DESCRIPTION, and from the CLAIMS.

### BRIEF DESCRIPTION OF THE DRAWINGS

The invention will hereinafter be described in conjunction with the appended drawings, where like designations denote like elements, and:

FIG. 1 is a graphical representation of tensile strength testing data of a control group sample.

FIG. 2 is a graphical representation of tensile strength testing data a first embodiment of a hair dye removal composition without arginine or cysteine.

FIG. 3 is a graphical representation of tensile strength testing data of a second embodiment of a hair dye removal composition comprising arginine.

FIG. 4 is a graphical representation of tensile strength testing data of a third embodiment of a hair dye removal composition comprising cysteine.

FIG. 5 is a graphical representation of tensile strength testing data of a fourth embodiment of a hair dye removal composition comprising both arginine and cysteine.

### DESCRIPTION

This disclosure, its aspects and implementations, are not limited to the specific components or assembly procedures disclosed herein. Many additional components and assembly procedures known in the art consistent with the intended hair dye removal compositions and/or mixing procedures for a hair dye removal composition will become apparent for use with implementations of hair dye removal compositions from this disclosure. Accordingly, for example, although particular hair dye removal compositions are disclosed, such hair dye removal compositions and implementing components may comprise any shape, size, style, type, model, version, measurement, concentration, material, quantity, and/or the like as is known in the art for such hair dye removal compositions and implementing components, consistent with the intended use of a hair dye removal composition.

Contemplated as part of this disclosure are various embodiments of a hair dye removal composition that may be used for cleansing and/or conditioning the hair of a user as well as for repairing damage to the hair that has been caused by chemical treatments. According to some aspects, the compositions described herein may be used to remove build up and coatings from hair, metal salts and other minerals, temporary and semi-permanent hair dye, hairspray, and henna. Compositions described herein may also be used for the treatment of damaged, dried-out hairs, as well as softening and removal of excess permanent hair dye color when mixed with hydrogen peroxide. Various embodiments described herein are advantageous to conventional hair dye removal compositions because they allow a user to switch hair color shades more easily, the composition is safe and gentle to use every time a user re-colors his/her hair, the composition removes permanent and semi-permanent hair color without bleach or ammonia, the composition repairs damaged hair, and/or the composition removes styling products from hair. In some embodiments, the compositions are devoid of triethanolamine, making the composition safer for users.

As used herein, “composition” is a term used in its broadest sense and may refer to a mixture of constituent substances or ingredients. Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

It is understood that the disclosed invention is not limited to the particular methodology, protocols and materials described as these can vary. It is also understood that the terminology used herein is for the purposes of describing particular embodiments only and is not intended to limit the scope of the present invention, which will be limited only by the appended claims. Those skilled in the art will recognize, or be able to ascertain using no more than routine experimentation, many equivalents to the specific embodiments of the invention described herein. Such equivalents are intended to be encompassed by the following disclosure.

One or more embodiments of a hair dye removal composition comprise a pH buffer. The pH buffer may comprise any pH buffer known in the art for hair dye removal compositions, such as but not limited to amp-95 2-amino-2-methyl-1-propanol, floraester K-20W hydrolyzed jojoba esters and water, and the like. In some, non-limiting embodiments, the pH buffer comprises aminomethyl propanol. The amount of pH buffer in various hair dye removal compositions may vary. According to some aspects, the pH buffer comprises between approximately 1 wt % and 7 wt % of the hair dye removal composition. In some embodiments, the pH buffer may comprise between approximately 1.5 wt % and approximately 6 wt % of the hair dye removal composition, between approximately 2 wt % and approximately 5 wt % of the hair dye removal composition, between approximately 2.5 wt % and approximately 4 wt % of the hair dye removal composition, between approximately 2.5 wt % and approximately 3.5 wt % of the hair dye removal composition, or between approximately 2.75 wt % and 3.25 wt % of the hair dye removal composition. In a non-limiting embodiment, the pH buffer comprises approximately 3 wt % of the hair dye removal composition, and the pH buffer may comprise aminomethyl propanol.

One or more embodiments of a hair dye removal composition comprise at least one cleansing agent. The cleansing agent may comprise any cleansing agent known in the art, and may simultaneously cleanse hair, emulsify the hair dye removal composition, and act as a humectant. In some non-limiting embodiments, the cleansing agent may comprise an oil, such as but not limited to sulfated castor oil. The amount of cleansing agent in various hair dye removal compositions may vary. According to some aspects, the cleansing agent comprises between approximately 50 wt % and 90 wt % of the hair dye removal composition. In some embodiments, the cleansing agent may comprise between approximately 60 wt % and approximately 80 wt % of the hair dye removal composition, between approximately 65 wt % and approximately 75 wt % of the hair dye removal composition, between approximately 68 wt % and approximately 74 wt % of the hair dye removal composition, between approximately 70 wt % and approximately 72 wt % of the hair dye removal composition, or between approximately 70.5 wt % and 71.5 wt % of the hair dye removal composition. In a non-limiting embodiment, the hair cleansing agent comprises approximately 71 wt % of the hair dye removal composition, and the cleansing agent may comprise sulfated castor oil.

One or more embodiments of a hair dye removal composition comprise at least one humectant. The humectant may comprise any humectant known in the art, such as but not limited to triethylene glycol, propylene glycol, tripropylene glycol, other polypropylene glycols, glycerin, sorbitol, hexylene and butylene glycol, urea, and collagen. The humectant may be adapted to repair the hair of the user after treatment by the hair dye removal composition.

According to some aspects, the humectant may comprise between approximately 5 wt % and approximately 30 wt % of the hair dye removal composition. In some embodiments, the humectant may comprise between approximately 7.5 wt % and approximately 25 wt % of the hair dye removal composition, between approximately 10 wt % and approximately 20 wt % of the hair dye removal composition, between approximately 12 wt % and approximately 19 wt % of the hair dye removal composition, between approximately 14 wt % and approximately 18 wt % of the hair dye removal composition, or between approximately 15 wt % and 16.5 wt % of the hair dye removal composition. In a non-limiting embodiment, humectant comprises approximately 15.75 wt % of the hair dye removal composition, and the humectant may comprise propylene glycol. In other embodiments, the humectant may comprise approximately 15 wt % of the hair dye removal composition.

One or more embodiments of a hair dye removal composition comprise at least one rheology modifier. The rheology modifier may comprise any rheology modifier known in the art adapted to modify the rheology, thickness, and/or viscosity of the hair dye removal composition. The rheology modifier may include but is not limited to acrylates/steareth-20 methacrylate copolymer, guar gum, supercol U, and/or xantham gum. According to some aspects, the rheology modifier may comprise between approximately 0.5 wt % and approximately 10 wt % of the hair dye removal composition. In some embodiments, the rheology modifier may comprise between approximately 2 wt % and approximately 8 wt % of the hair dye removal composition, between approximately 3 wt % and approximately 7 wt % of the hair dye removal composition, between approximately 4 wt % and approximately 6 wt % of the hair dye removal composition, between approximately 4.5 wt % and approximately 5.5 wt % of the hair dye removal composition, or between approximately 4.75 wt % and 5.25 wt % of the hair dye removal composition. In a non-limiting embodiment, rheology modifier comprises approximately 5 wt % of the hair dye removal composition, and the rheology modifier may comprise acrylates/steareth-20 methacrylate copolymer.

One or more embodiments of a hair dye removal composition comprise at least one acid. The acid may comprise any acid or other chemical compound known in the art adapted to modify the pH of the composition and/or decrease inflammation or prevent breakouts on the scalp of the user. The acid may comprise but is not limited to salicylic acid. According to some aspects, the acid may comprise between approximately 0.5 wt % and approximately 6 wt % of the hair dye removal composition. In some embodiments, the acid may comprise between approximately 1 wt % and approximately 5.5 wt % of the hair dye removal composition, between approximately 1.5 wt % and approximately 5 wt % of the hair dye removal composition, between approximately 2 wt % and approximately 4 wt % of the hair dye removal composition, between approximately 2.5 wt % and approximately 3.5 wt % of the hair dye removal composition, or between approximately 2.75 wt % and 3.25 wt % of the hair dye removal composition. In a non-limiting embodiment, acid comprises approximately 3 wt % of the hair dye removal composition, and the acid may comprise salicylic acid. In other embodiments, the acid comprises approximately 4.75 wt % of the hair dye removal composition.

One or more embodiments of a hair dye removal composition comprise water. The water used in the hair dye removal composition may comprise deionized water, distilled water, or any other available water. According to some aspects, the water may comprise between approximately 0.5 wt % and approximately 6 wt % of the hair dye removal composition. In some embodiments, the water may comprise between approximately 1 wt % and approximately 5.5 wt % of the hair dye removal composition, between approximately 1.25 wt % and approximately 5 wt % of the hair dye removal composition, between approximately 1.5 wt % and approximately 4 wt % of the hair dye removal composition, between approximately 1.75 wt % and approximately 3 wt % of the hair dye removal composition, or between approximately 2 wt % and 2.25 wt % of the hair dye removal composition. In a non-limiting embodiment, water comprises approximately 2.07 wt % of the hair dye removal composition, and the water may comprise deionized water.

One or more embodiments of a hair dye removal composition comprise a preservative. The preservative may comprise any preservative known in the art, such as but not limited to ethylenediaminetetraacetic acid (EDTA). According to some aspects, the preservative may comprise between approximately 0.01 wt % and approximately 0.2 wt % of the hair dye removal composition. In some embodiments, the preservative may comprise between approximately 0.02 wt % and approximately 0.1 wt % of the hair dye removal composition, between approximately 0.03 wt % and approximately 0.09 wt % of the hair dye removal composition, between approximately 0.04 wt % and approximately 0.08 wt % of the hair dye removal composition, or between approximately 0.05 wt % and approximately 0.07 wt % of the hair dye removal composition. In a non-limiting embodiment, preservative comprises approximately 0.06 wt % of the hair dye removal composition, and the preservative may comprise EDTA.

One or more embodiments of a hair dye removal composition may comprise a fragrance. The fragrance may comprise any fragrance known in the art. According to some aspects, the fragrance comprises between approximately 0.05 wt % and approximately 0.25 wt % of the hair dye removal composition. In some embodiments, the fragrance comprises between approximately 0.06 wt % and approximately 0.24 wt % of the hair dye removal composition, between approximately 0.07 wt % and approximately 0.21 wt % of the hair dye removal composition, between approximately 0.08 wt % and approximately 0.18 wt % of the hair dye removal composition, between approximately 0.09 wt % and approximately 0.15 wt % of the hair dye removal composition, or between approximately 0.10 wt % and approximately 0.14 wt % of the hair dye removal composition. In a non-limiting embodiment, fragrance comprises approximately 0.12 wt % of the hair dye removal composition.

One or more embodiments of a hair dye removal composition may comprise arginine. According to some aspects, the arginine comprises between approximately 0.05 wt % and approximately 2.0 wt % of the hair dye removal composition. In some embodiments, the arginine comprises between approximately 0.03 wt % and approximately 1.0 wt % of the hair dye removal composition, between approximately 0.05 wt % and approximately 0.8 wt % of the hair dye removal composition, between approximately 0.08 wt % and approximately 0.7 wt % of the hair dye removal composition, between approximately 0.1 wt % and approximately 0.5 wt % of the hair dye removal composition, or between approximately 0.2 wt % and approximately 0.4 wt % of the hair dye removal composition. In a non-limiting embodiment, arginine comprises approximately 0.12 wt % of the hair dye removal composition.

One or more embodiments of a hair dye removal composition may comprise cysteine. According to some aspects, the cysteine comprises between approximately 0.01 wt % and approximately 0.2 wt % of the hair dye removal composition. In some embodiments, the preservative may comprise between approximately 0.01 wt % and approximately 0.1 wt % of the hair dye removal composition, between approximately 0.015 wt % and approximately 0.09 wt % of the hair dye removal composition, between approximately 0.02 wt % and approximately 0.08 wt % of the hair dye removal composition, or between approximately 0.025 wt % and approximately 0.05 wt % of the hair dye removal composition. In a non-limiting embodiment, preservative comprises approximately 0.03 wt % of the hair dye removal composition.

In embodiments of a hair dye removal composition that contain both arginine and cysteine, in addition to removing hair dye and/or substance buildup on the hair, these embodiments also achieve an unexpected level of hair strengthening as demonstrated by the following test results. Tensile testing is a common practice among manufacturers of various hair products such as shampoos, dyes, and gels. Here, large sample sizes were used and tested by Instron® tensile testing equipment. The study tested a control sample of hair that did not have any products or compositions applied to it, hair washed with an embodiment of the hair dye removal composition comprising no arginine or cysteine, hair washed with an embodiment of the hair dye removal composition comprising arginine, hair washed with an embodiment of the hair dye removal composition comprising cysteine, and hair washed with an embodiment of the hair dye removal composition comprising both arginine and cysteine.

Table 1, below summarizes the tensile testing results of the control sample. The hair was bleached and colored for 30 minutes. The hair was washed one, two, and three times with no product applied to the hair.

TABLE 1

| | | % Damaged | % Damaged | % Damaged |
|---|---|---|---|---|
| | Tensile | Hair | Hair | Hair |
| | Stress | Three | Two | One |
| | (mpa) | Washes | Washes | Wash |
| Hair tested with | 0 | 0 | 0 | 0 |
| no product applied | | | | |
| (control sample) | | | | |
| Hair tested with | 50 | 25 | 27 | 29 |
| no product applied | | | | |
| (control sample) | | | | |
| Hair tested with | 100 | 35 | 37 | 39 |
| no product applied | | | | |
| (control sample) | | | | |
| Hair tested with | 150 | 60 | 65 | 67 |
| no product applied | | | | |
| (control sample) | | | | |
| Hair tested with | 200 | 100 | 100 | 100 |
| no product applied | | | | |
| (control sample) | | | | |
| Hair tested with | 250 | 100 | 100 | 100 |
| no product applied | | | | |
| (control sample) | | | | |
| Hair tested with | 300 | 100 | 100 | 100 |
| no product applied | | | | |
| (control sample) | | | | |

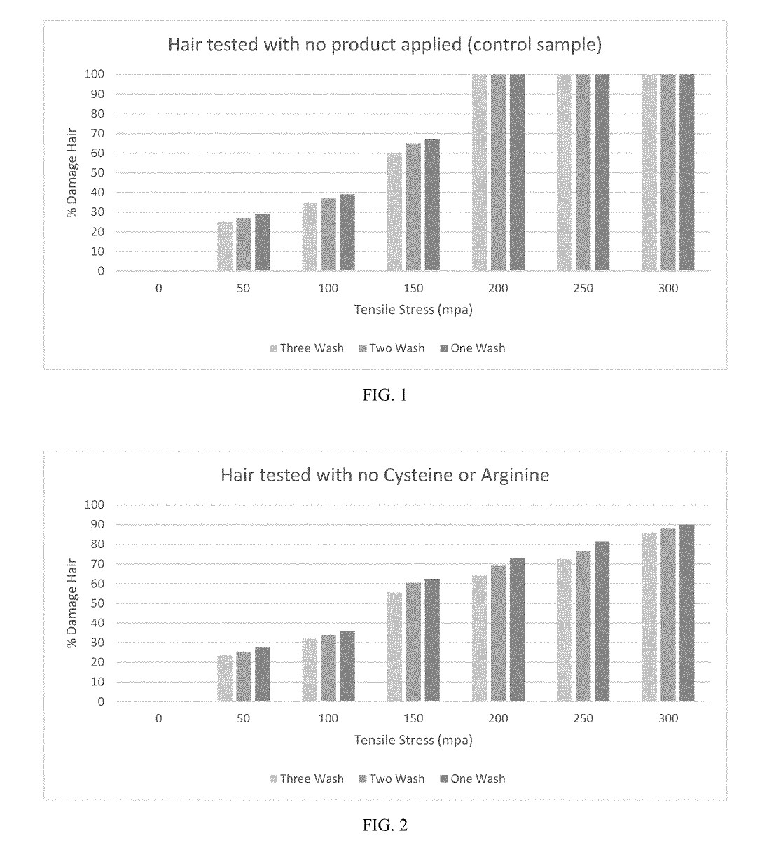

FIG. 1 provides a graphical depiction of the data of Table 1. As shown, after one, two, or three washings, 100% of the hair failed at 200 mPa.

Table 2, below summarizes the tensile testing results of one embodiment of a hair dye removal composition. The hair was bleached and colored for 30 minutes. The hair was washed one, two, and three times with an embodiment of the hair dye removal composition comprising sulfated castor oil, propylene glycol, acrylates/steareth-20 methacrylate copolymer, salicylic acid, and aminomethyl propanol. This embodiment of the composition did not contain any cysteine or arginine.

TABLE 2

| | | % Damaged | % Damaged | % |
|---|---|---|---|---|
| | Tensile | Hair | Hair | Damaged |
| | Stress | Three | Two | Hair |
| | (mpa) | Washes | Washes | One Wash |
| Hair tested with no | 0 | 0 | 0 | 0 |
| Cysteine or Arginine | | | | |
| Hair tested with no | 50 | 23.5 | 25.5 | 27.5 |
| Cysteine or Arginine | | | | |
| Hair tested with no | 100 | 32 | 34 | 36 |
| Cysteine or Arginine | | | | |
| Hair tested with no | 150 | 55.5 | 60.5 | 62.5 |
| Cysteine or Arginine | | | | |
| Hair tested with no | 200 | 64 | 69 | 73 |
| Cysteine or Arginine | | | | |
| Hair tested with no | 250 | 72.5 | 76.5 | 81.5 |
| Cysteine or Arginine | | | | |
| Hair tested with no | 300 | 86 | 88 | 90 |
| Cysteine or Arginine | | | | |

FIG. 2 provides a graphical depiction of the data of Table 2. As shown, after one, washing, 90% of the hair failed, after two washings, 88% of the hair failed and after three washings, 86% of the hair failed at 300 mPa.

Table 3, below summarizes the tensile testing results of one embodiment of a hair dye removal composition containing arginine. The hair was bleached and colored for 30 minutes. The hair was washed one, two, and three times with an embodiment of the hair dye removal composition comprising sulfated castor oil, propylene glycol, acrylates/steareth-20 methacrylate copolymer, salicylic acid, aminomethyl propanol, and arginine.

TABLE 3

| | | % Damaged | % Damaged | % |
|---|---|---|---|---|
| | Tensile | Hair | Hair | Damaged |
| | Stress | Three | Two | Hair |
| | (mpa) | Washes | Washes | One Wash |
| Hair tested with only | 0 | 0 | 0 | 0 |
| Arginine | | | | |
| Hair tested with only | 50 | 22 | 24 | 26 |
| Arginine | | | | |
| Hair tested with only | 100 | 29 | 31 | 33 |
| Arginine | | | | |
| Hair tested with only | 150 | 51 | 56 | 58 |
| Arginine | | | | |
| Hair tested with only | 200 | 58 | 63 | 67 |
| Arginine | | | | |
| Hair tested with only | 250 | 65 | 69 | 74 |
| Arginine | | | | |
| Hair tested with only | 300 | 77 | 79 | 81 |
| Arginine | | | | |

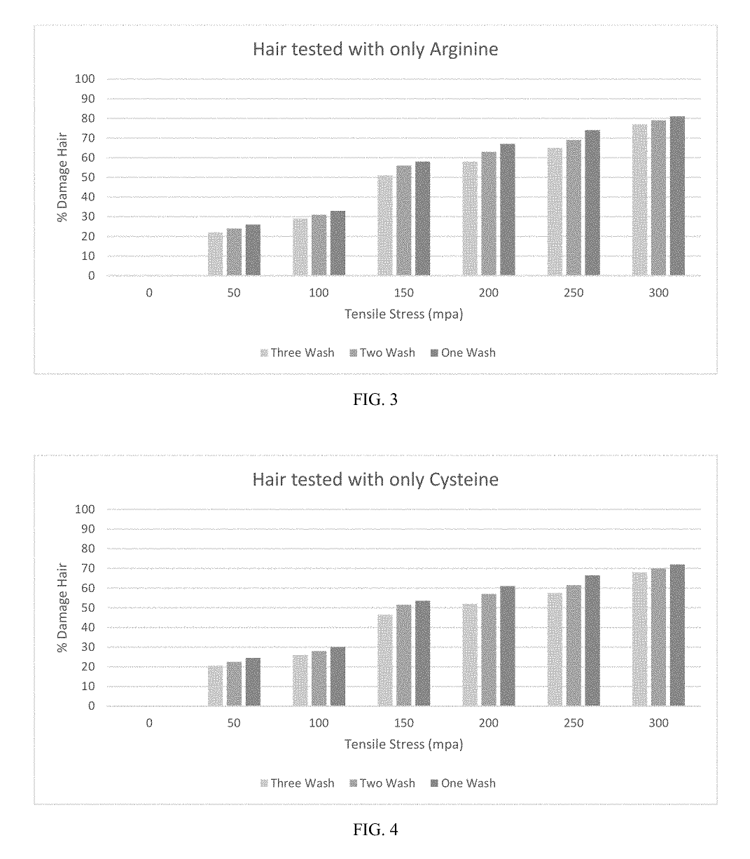

FIG. 3 provides a graphical depiction of the data of Table 3. As shown, after one, washing, 81% of the hair failed, after two washings, 79% of the hair failed and after three washings, 77% of the hair failed at 300 mPa.

Table 4, below summarizes the tensile testing results of one embodiment of a hair dye removal composition containing cysteine. The hair was bleached and colored for 30 minutes. The hair was washed one, two, and three times with an embodiment of the hair dye removal composition comprising sulfated castor oil, propylene glycol, acrylates/steareth-20 methacrylate copolymer, salicylic acid, aminomethyl propanol, and cysteine.

TABLE 4

| | | % Damaged | % Damaged | % |
|---|---|---|---|---|
| | Tensile | Hair | Hair | Damaged |
| | Stress | Three | Two | Hair |
| | (mpa) | Washes | Washes | One Wash |
| Hair tested with only | 0 | 0 | 0 | 0 |
| Cysteine | | | | |
| Hair tested with only | 50 | 20.5 | 22.5 | 24.5 |
| Cysteine | | | | |
| Hair tested with only | 100 | 26 | 28 | 30 |
| Cysteine | | | | |
| Hair tested with only | 150 | 46.5 | 51.5 | 53.5 |
| Cysteine | | | | |
| Hair tested with only | 200 | 52 | 57 | 61 |
| Cysteine | | | | |
| Hair tested with only | 250 | 57.5 | 61.5 | 66.5 |
| Cysteine | | | | |
| Hair tested with only | 300 | 68 | 70 | 72 |
| Cysteine | | | | |

FIG. 4 provides a graphical depiction of the data of Table 4. As shown, after one, washing, 72% of the hair failed, after two washings, 70% of the hair failed and after three washings, 68% of the hair failed at 300 mPa.

Table 5, below summarizes the tensile testing results of one embodiment of a hair dye removal composition containing a combination of both arginine and cysteine. The hair was bleached and colored for 30 minutes. The hair was washed one, two, and three times with an embodiment of the hair dye removal composition comprising sulfated castor oil, propylene glycol, acrylates/steareth-20 methacrylate copolymer, salicylic acid, aminomethyl propanol, arginine and cysteine.

TABLE 5

| | | % Damaged | % Damaged | % |
|---|---|---|---|---|
| | Tensile | Hair | Hair | Damaged |
| | Stress | Three | Two | Hair |
| | (mpa) | Washes | Washes | One Wash |
| Hair tested with | 0 | 0 | 0 | 0 |
| Arginine and Cysteine | | | | |
| Hair tested with | 50 | 11 | 13 | 15 |
| Arginine and Cysteine | | | | |
| Hair tested with | 100 | 16 | 18 | 20 |
| Arginine and Cysteine | | | | |
| Hair tested with | 150 | 24 | 29 | 31 |
| Arginine and Cysteine | | | | |
| Hair tested with | 200 | 26 | 31 | 35 |
| Arginine and Cysteine | | | | |
| Hair tested with | 250 | 28.5 | 32.5 | 37.5 |
| Arginine and Cysteine | | | | |
| Hair tested with | 300 | 39 | 41 | 43 |
| Arginine and Cysteine | | | | |

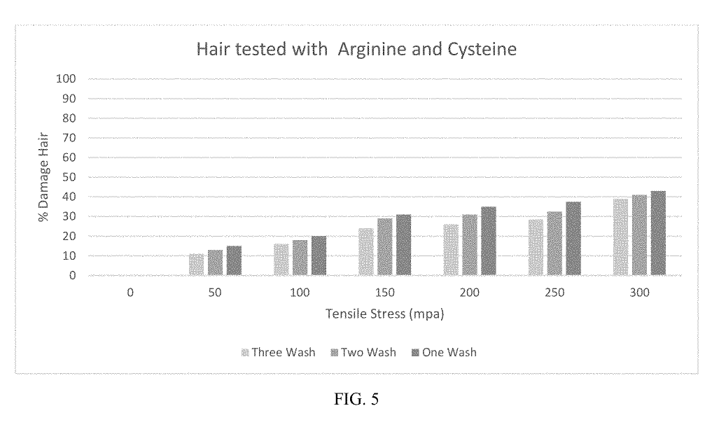

FIG. 5 provides a graphical depiction of the data of Table 5. As shown, after one, washing, only 43% of the hair failed, after two washings, only 41% of the hair failed and after three washings, only 39% of the hair failed at 300 mPa. The data therefore supports the finding of an unexpected level of hair strengthening resulting from the addition of both arginine and cysteine into the hair dye removal composition.

Also contemplated as part of this disclosure is a method of preparing or mixing a hair dye removal composition for use as a hair color removal and/or a hair strengthening composition. The method may utilize any of the wt % of ingredients or materials described elsewhere in this application. For example, a method may comprise heating a cleansing agent. Unless otherwise specified, the cleansing agent heated in this step may comprise any of the cleansing agents described above, such as but not limited to sulfated castor oil, and the amount of cleansing agent heated may comprise any amount of cleansing agent sufficient to satisfy the above referenced wt % of the hair dye removal composition, such as but not limited to between approximately 50 wt % and 90 wt % of the hair dye removal composition; between approximately 60 wt % and approximately 80 wt % of the hair dye removal composition, between approximately 65 wt % and approximately 75 wt % of the hair dye removal composition, between approximately 68 wt % and approximately 74 wt % of the hair dye removal composition, between approximately 70 wt % and approximately 72 wt % of the hair dye removal composition, or between approximately 70.5 wt % and 71.5 wt % of the hair dye removal composition.

A method of preparing a hair dye removal composition may comprise adding a cleansing agent, such as but not limited to sulfated castor oil, to a batch tank or other mixing container. A method may further comprise starting a batch tank mixer at a rate that turns over the cleansing agent without incorporating air into the cleansing agent. For example, the batch tank mixer may be set at approximately 30 rpm, 35 rpm, 40 rpm, 45 rpm, 50 rpm, 55 rpm, or 60 rpm. A method may further comprise heating the cleansing agent with any suitable heater known in the art, such as but not limited to a steam jacket. The cleansing agent may be heated to approximately 30-35 degrees Celsius, approximately 35-40 degrees Celsius, approximately 40-45 degrees Celsius, approximately 45-50 degrees Celsius approximately 50-55 degrees Celsius, approximately 55-60 degrees Celsius, approximately 60-65 degrees Celsius, or approximately 65-70 degrees Celsius.

A method of preparing a hair dye removal composition may further comprise adding a preservative, such as but not limited to EDTA, to the cleansing agent. The preservative may be added to the cleansing agent before any other ingredients or compounds are added to the cleansing agent. In a non-limiting embodiment, adding the preservative to the cleansing agent comprises sprinkling the preservative into the cleansing agent in the batch tank. A method may further comprise mixing the cleansing agent and the preservative for a minimum of 5 minutes, 10 minutes, 15 minutes, 20 minutes, 25 minutes, 30 minutes, 35 minutes, 40 minutes, 45 minutes, 50 minutes, 55 minutes, or 60 minutes.

A method of preparing a hair dye removal composition may further comprise premixing a first portion of a humectant, such as but not limited to propylene glycol, and a first portion of an acid, such as but not limited to salicylic acid. The first portion of the humectant may comprise approximately ¼, ⅓, ½, ⅔, or ¾ of the total amount of humectant in the final hair dye removal composition. The first portion of the acid may comprise approximately ¼, ⅓, ½, ⅔, or ¾ of the total amount of acid in the final hair dye removal composition.

A method of preparing a hair dye removal composition may further comprise premixing the first portion of the humectant and the first portion of the acid at a rate that turns over the mixture without incorporating air into the mixture. A method may further comprise heating the premixture of the first portion of the humectant and the first portion of the acid to approximately 30-35 degrees Celsius, approximately 35-40 degrees Celsius, approximately 40-45 degrees Celsius, approximately 45-50 degrees Celsius approximately 50-55 degrees Celsius, approximately 55-60 degrees Celsius, approximately 60-65 degrees Celsius, or approximately 65-70 degrees Celsius. A method may further comprise mixing the premixture of the first portion of the humectant and the first portion of the acid for 5 minutes, 10 minutes, 15 minutes, 20 minutes, 25 minutes, 30 minutes, 35 minutes, 40 minutes, 45 minutes, 50 minutes, 55 minutes, 60 minutes, and/or until the premixture is clear. A method may further comprise adding the premixture of the first portion of the humectant and the first portion of the acid to the cleansing agent.

A method of preparing a hair dye removal composition may further comprise premixing a second portion of a humectant, such as but not limited to propylene glycol, and a second portion of an acid, such as but not limited to salicylic acid. The second portion of the humectant may comprise approximately ¼, ⅓, ½, ⅔, or ¾ of the total amount of humectant in the final hair dye removal composition. The second portion of the acid may comprise approximately ¼, ⅓, ½, ⅔, or 3/4 of the total amount of acid in the final hair dye removal composition.

A method of preparing a hair dye removal composition may further comprise premixing the second portion of the humectant and the second portion of the acid at a rate that turns over the mixture without incorporating air into the mixture. A method may further comprise heating the premixture of the second portion of the humectant and the second portion of the acid to approximately 30-35 degrees Celsius, approximately 35-40 degrees Celsius, approximately 40-45 degrees Celsius, approximately 45-50 degrees Celsius approximately 50-55 degrees Celsius, approximately 55-60 degrees Celsius, approximately 60-65 degrees Celsius, or approximately 65-70 degrees Celsius. A method may further comprise mixing the premixture of the second portion of the humectant and the second portion of the acid for 5 minutes, 10 minutes, 15 minutes, 20 minutes, 25 minutes, 30 minutes, 35 minutes, 40 minutes, 45 minutes, 50 minutes, 55 minutes, 60 minutes, and/or until the premixture is clear. A method may further comprise adding the premixture of the second portion of the humectant and the second portion of the acid to the cleansing agent and the premixture of the first portion of the humectant and the first portion of the acid.

A method may further comprise mixing the cleansing agent, the humectant, the acid, and the preservative for 5 minutes, 10 minutes, 15 minutes, 20 minutes, 25 minutes, 30 minutes, 35 minutes, 40 minutes, 45 minutes, 50 minutes, 55 minutes, 60 minutes, and/or until the new mixture is clear. The speed of the mixer may be increased to produce a turbulent batch surface without creating excessive splashing. A method may further comprise heating the mixture of the cleansing agent, the humectant, the acid, and the preservative to approximately 30-35 degrees Celsius, approximately 35-40 degrees Celsius, approximately 40-45 degrees Celsius, approximately 45-50 degrees Celsius approximately 50-55 degrees Celsius, approximately 55-60 degrees Celsius, approximately 60-65 degrees Celsius, or approximately 65-70 degrees Celsius.

A method may further comprise adding a rheology modifier to the mixture of the cleansing agent, the humectant, the acid, and the preservative. As noted above, the rheology modifier may comprise acrylates/steareth-20 methacrylate copolymer or any other rheology modifier known in the art. In some embodiments, it is advantageous to slowly add the rheology modifier to the mixture of the cleansing agent, the humectant, the acid, and the preservative. For example, the rheology modifier may be added at a maximum rate of 10 kg/minute, 15 kg/minute, 20 kg/minute, 25 kg/minute, or 30 kg/minute. In some embodiments, the rheology modifier is added at a maximum rate of 24 kg/minute. If the rheology modifier is added too quickly an undesirable precipitation will form.

A method may further comprise adding water to the mixture of the cleansing agent, the humectant, the acid, the preservative, and the rheology modifier. More particularly, a method may comprise adding hot water to the batch mixture of the cleansing agent, the humectant, the acid, the preservative, and the rheology modifier through, for example, the spray ball. The batch may be covered when water is added to the mixture.

A method may further comprise mixing one or more of cysteine and arginine with water until dissolved. The one or more of the cysteine and the arginine may be of a powdered or liquid form. The one or more of the cysteine and arginine may be mixed in a pre-mix phase and the water may be at a temperature range of approximately 10-40 degrees Celsius, approximately 15-35 degrees Celsius, or approximately 20-30 degrees Celsius. It is preferable not to heat the cysteine and/or arginine to a high temperature such as approximately 60 degrees Celsius or above as such a high level of heat may destroy these amino acids. Thus, the premixed one or more of the cysteine and the arginine solution may be added at or near the end of a batch cycle when the composition has cooled to a sufficient level to prevent destruction of the amino acid(s).

A method may further comprise mixing the cleansing agent, the humectant, the acid, the preservative, the rheology modifier, one or more of the cysteine and the arginine, and the water.

According to some aspects, a method comprises mixing the batch of the cleansing agent, the humectant, the acid, the preservative, the rheology modifier, and the water for a minimum of 15 minutes, 30 minutes, 45 minutes, 60 minutes, 75 minutes, or 90 minutes. A method may further comprise testing the mixture of the cleansing agent, the humectant, the acid, the preservative, the rheology modifier, and the water by transferring a sample of the mixture to a clear glass jar and allowing the sample to de-aerate. A method may further comprise examining if white droplets are still present, the method comprises continuing to mix until the droplets dissolve completely. A method may further comprise adjusting the mixer to turn the mixture over without excessive aeration.

A method may further comprise adding aminomethyl propanol to the mixture of the cleansing agent, the humectant, the acid, the preservative, the rheology modifier, and the water. A method may further comprise adding a fragrance to the mixture of the cleansing agent, the humectant, the acid, the preservative, the rheology modifier, the water, and the aminomethyl propanol. A method may further comprise mixing the cleansing agent, the humectant, the acid, the preservative, the rheology modifier, the water, the aminomethyl propanol, and the fragrance for a minimum of 5 minutes, 10 minutes, 15 minutes, 20 minutes, 25 minutes 30 minutes, 35 minutes, 40 minutes, or 45 minutes to form the hair dye removal composition. A method may further comprise inserting the hair dye removal composition into a sanitized container or a container for sanitization.

Also contemplated herein is a method of using any of the embodiments of hair dye removal compositions described or otherwise contemplated herein. A user may use a hair dye removal composition to remove build up and coating from his/hair. A method may comprise applying a sufficient amount of a hair dye removal composition to the hair of a user. The hair treatment applied may be undiluted. A method may further comprise massaging the hair dye removal composition into the hair of the user to saturate the hair with the hair dye removal composition. A method may further comprise covering the hair of the user with a plastic cap for 10 minutes, 15 minutes, 20 minutes, 25 minutes, 30 minutes, 35 minutes, 40 minutes, or at least 45 minutes after the hair has been saturated with the hair dye removal composition. A method may further comprise rinsing the hair dye removal composition from the hair of the user. In some embodiments, a method comprises diluting the hair dye removal composition with 20v peroxide.

Once diluted, the hair dye removal composition and the peroxide may comprise substantially equal parts. When using peroxide-diluted hair dye removal composition, a plastic cap may be used to cover the hair of the user for less time than when using an undiluted hair dye removal composition.

It will be understood that implementations are not limited to the specific components disclosed herein, as virtually any components consistent with the intended operation of a method and/or system implementation for hair dye removal compositions may be utilized.

Accordingly, for example, although particular cleansing agents, humectants, rheology modifiers, preservatives, and fragrances may be disclosed, such components may comprise any shape, size, style, type, model, version, class, grade, measurement, concentration, material, weight, quantity, and/or the like consistent with the intended operation of a method and/or system implementation for a hair dye removal composition may be used.

In places where the description above refers to particular implementations of hair dye removal compositions, it should be readily apparent that a number of modifications may be made without departing from the spirit thereof and that these implementations may be applied to other hair dye removal compositions. The accompanying claims are intended to cover such modifications as would fall within the true spirit and scope of the disclosure set forth in this document. The presently disclosed implementations are, therefore, to be considered in all respects as illustrative and not restrictive, the scope of the disclosure being indicated by the appended claims rather than the foregoing description. All changes that come within the meaning of and range of equivalency of the claims are intended to be embraced therein.

1. A hair dye removal composition, comprising a cleansing agent, a humectant, arheology modifier, arginine, cysteine, and aminomethyl propanol.

2. The hair dye removal composition of claim 1, wherein the cleansing agent comprises between approximately 60 wt % to approximately 80 wt % of the hair dye removal composition, the humectant comprises between approximately 10 wt % to approximately 20 wt % of the hair dye removal composition, the rheology modifier comprises between approximately 2 wt % and approximately 10 wt % of hair dye removal composition, the arginine comprises between approximately 0.05 wt % and approximately 0.8 wt % of the hair dye removal composition, the cysteine comprises between approximately 0.02 wt % and approximately 0.08 wt % of the hair dye removal composition, and the aminomethyl propanol comprises between approximately 1 wt % and approximately 6 wt % of the hair dye removal composition.

3. The hair dye removal composition of claim 2, further comprising salicylic acid, the salicylic acid comprising between approximately 1 wt % and approximately 6 wt % of the hair dye removal composition.

4. The hair dye removal composition of claim 3, wherein the cleansing agent comprises between approximately 68 wt % and approximately 74 wt % of the hair dye removal composition, the humectant comprises between approximately 14 wt % to approximately 17 wt % of the hair dye removal composition, the rheology modifier comprises between approximately 4 wt % and approximately 6 wt % of hair dye removal composition, the aminomethyl propanol comprises between approximately 2 wt % and approximately 4 wt % of the hair dye removal composition, the arginine comprises between approximately 0.03 wt % and approximately 0.1 wt % of the hair dye removal composition, the cysteine comprises between approximately 0.01 wt % and approximately 0.1 wt % of the hair dye removal composition, and the salicylic acid comprises between approximately 2 wt % and approximately 4 wt % of the hair dye removal composition.

5. The hair dye removal composition of claim 4, wherein the cleansing agent comprises sulfated castor oil, the humectant comprises propylene glycol, and the rheology modifier comprises acrylates/steareth-20 methacrylate copolymer.

6. The hair dye removal composition of claim 5, further comprising water and a preservative, the water comprising between approximately 1 wt % and approximately 3 wt % of the hair dye removal composition and the preservative comprising between approximately 0.01 wt % and approximately 0.1 wt % of the hair dye removal composition.

7. The hair dye removal composition of claim 6, further comprising a fragrance, the fragrance comprising between approximately 0.05 wt % and approximately 0.25 wt % of the hair conditioning composition.

8. A hair dye removal composition, comprising: oil, humectant, arginine, cysteine, and a pH buffer selected from the group consisting of 2-amino-2-methyl-1-propanol and aminomethyl propanol, the oil comprising greater than 50 wt % of the hair dye removal composition, the humectant comprising less than 20 wt % of the hair dye removal composition, the arginine comprising less than 5 wt % of the hair dye removal composition, the cysteine comprising less than 1 wt %and the pH buffer comprising less than 5 wt % of the hair dye removal composition.

9. The hair dye removal composition of claim 8, further comprising a rheology modifier and an acid, the rheology modifier comprising less than 10 wt % of the hair dye removal composition and the acid comprising less than 5 wt % of the hair dye removal composition.

10. The hair dye removal composition of claim 9, wherein the rheology modifier comprises acrylates/steareth-20 methacrylate copolymer and the acid comprises salicylic acid.

11. The hair dye removal composition of claim 8, wherein the oil comprises between approximately 60 wt % to approximately 80 wt % of the hair dye removal composition, the humectant comprises between approximately 10 wt % to approximately 20 wt % of the hair dye removal composition, and the aminomethyl propanol comprises between approximately 2 wt% and approximately 4 wt % of the hair dye removal composition.

12. The hair dye removal composition of claim 8, wherein the oil comprises between approximately 68 wt % and approximately 74 wt % of the hair dye removal composition, the humectant comprises between approximately 14 wt % to approximately 17 wt % of the hair dye removal composition, and the aminomethyl propanol comprises between approximately 2.5 wt % and approximately 3.5 wt % of the hair dye removal composition.

13. The hair dye removal composition of claim 8, wherein the oil comprises sulfated castor oil, the humectant comprises propylene glycol, and the pH buffer comprises amp-95 2-amino-2-methyl-1-propanol.

14. The hair dye removal composition of claim 8, further comprising water, a fragrance, and ethylenediaminetetraacetic acid (EDTA), the water comprising between approximately 1 wt % and approximately 3 wt % of the hair dye removal composition, the fragrance comprising less than 1 wt % of the hair dye removal composition, and the EDTA comprising less than 1 wt % of the hair dye removal composition.

15. A method of preparing a hair dye removal composition, comprising:
premixing arginine and cysteine in water;
heating a cleansing agent to between approximately 45 degrees Celsius and approximately 60 degrees Celsius;
mixing a humectant and a rheology modifier with the cleansing agent;
mixing aminomethyl propanol with the cleaning agent, the humectant, and the rheology modifier; and
mixing the premixed arginine and cysteine with the cleansing agent, the humectant, the rheology modifier, and the aminomethyl propanol at a temperature that is below 60 degrees Celsius.

16. The method of claim 15, wherein mixing the humectant and the rheology modifier with the cleansing agent comprises:
premixing a first portion of salicylic acid and a first portion of the humectant before mixing the first portion of the salicylic acid and the first portion of the humectant with the cleansing agent;
premixing a second portion of the salicylic acid and a second portion of the humectant before mixing the second portion of the salicylic acid and the second portion of the humectant with the cleansing agent, the first portion of the salicylic acid, and the first portion of the humectant; and
adding the rheology modifier to the first and second portions of the salicylic acid, the first and second portions of the humectant, and the cleansing agent at a maximum rate of 30 kg/minute.

17. The method of claim 16, further comprising:
mixing ethylenediaminetetraacetic acid (EDTA) with the cleansing agent for at least 15 minutes before mixing the first portion of the humectant and the first portion of the salicylic acid to the cleansing agent; and
adding water after adding the rheology modifier and before mixing the aminomethyl propanol with the cleansing agent.

18. The method of claim 17, further comprising mixing the cleansing agent, the EDTA, the first and second portions of the humectant, the first and second portions of the salicylic acid, the rheology modifier, and the water for at least 75 minutes before mixing the aminomethyl propanol with the cleansing agent.

19. The method of claim 18, wherein the arginine comprises between approximately 0.1 wt % and approximately 0.5 wt % of the hair dye removal composition, the cysteine comprises between approximately 0.01 wt % and approximately 0.1 wt % of the hair dye removal composition, the cleansing agent comprises between approximately 65 wt % and approximately 75 wt % of the hair dye removal composition, the EDTA comprises less than 1 wt % of the hair dye removal composition, the first portion of the humectant comprises between approximately 5 wt % and approximately 9 wt % of the hair dye removal composition, the first portion of the salicylic acid comprises between approximately 1.75 wt % and approximately 2.75 wt % of the hair dye removal composition, the second portion of the humectant comprises between approximately 5 wt % and approximately 9 wt % of the hair dye removal composition, the second portion of the salicylic acid comprises between approximately 1.75 wt % and approximately 2.75 wt % of the hair dye removal composition, the rheology modifier comprises between approximately 3 wt % and approximately 7 wt % of the hair dye removal composition, and the aminomethyl propanol comprises between approximately 2 wt % and approximately 4 wt % of the hair dye removal composition.

20. The method of claim 18, wherein the cleansing agent comprises sulfated castor oil, the humectant comprises propylene glycol, and the rheology modifier comprises acrylates/steareth-20 methacrylate copolymer.
